(19) United States
(12) Patent Application Publication
(10) Pub. No.: US 2010/0047803 A1
(43) Pub. Date: Feb. 25, 2010

(54) BIOMARKERS FOR ACETAMINOPHEN TOXICITY

(76) Inventors: Richard M. Weinshilboum, Rochester, MN (US); Brooke L. Fridley, Rochester, MN (US); Ann M. Moyer, Rochester, MN (US)

Correspondence Address: FISH & RICHARDSON P.C., PO BOX 1022, MINNEAPOLIS, MN, 55440-1022, US

(21) Appl. No.: 12/545,561

(22) Filed: Aug. 21, 2009

Related U.S. Application Data
(60) Provisional application No. 61/189,730, filed on Aug. 22, 2008.

(51) Int. Cl. C12Q 1/68

(52) U.S. Cl. 435/6

(57) ABSTRACT

Materials and methods for evaluating cellular responses to acetaminophen and assessing susceptibility to liver damage.

### CROSS-REFERENCE TO RELATED APPLICATIONS

This application claims priority to U.S. Provisional Application Ser. No. 61/189,730, filed on Aug. 22, 2008, which is incorporated by reference in its entirety herein.

### STATEMENT AS TO FEDERALLY SPONSORED RESEARCH

This invention was made with government support under grant nos. GM061388, GM35720, and GM028157, awarded by the National Institutes of Health. The government has certain rights in the invention.

### TECHNICAL FIELD

This document relates to materials and methods for assessing an individual's susceptibility to drug toxicity, and more particularly to materials and methods for assessing an individual's susceptibility to acetaminophen toxicity.

### BACKGROUND

Pharmacogenetics is the study of the role of inheritance in individual variation in response to drugs, nutrients and other xenobiotics. In the current post-genomic era, pharmacogenetics has evolved into pharmacogenomics (Wang et al. (2003) Pharmacogenetics 13:555-64; Weinshilboum and Wang (2004) Nature Rev. Drug Discovery 3:739-748; Guttmacher and Collins (2005) JAMA 294:1399-402; and Weinshilboum and Wang (2006) Annu. Rev. Genomics Hum. Genet. 7:223-45). Drug response phenotypes that are influenced by inheritance can vary from potentially life-threatening adverse reactions at one of the spectrum to lack of therapeutic efficacy at the other. The ability to determine whether and how a subject will respond to a particular drug can assist medical professionals in determining whether the drug should be administered to the subject, and at what dose.

A major challenge facing this component of individualized medicine is how to identify pharmacogenomically important candidate genes for a variety of drugs—including drugs yet to be developed—in an efficient and scientifically valid fashion. Clinical drug trials are expensive and require large patient populations. Academic centers can find it difficult to contribute to pharmacogenomic studies because of the size, complexity and cost of conducting trials to develop and test novel pharmacogenomic hypotheses. At the same time, the “blockbuster” drug approach that has been the major working model for pharmaceutical companies is increasingly challenged by the concept of “individualized” drug therapy. Thus, there is an increasing need to incorporate pharmacogenomics into drug development and early clinical trials. In addition, there is a great need for a model system that would represent common human genetic variation and that could be used to rapidly test drug response phenotypes.

Acetaminophen overdose is a major cause of acute hepatic failure. Inter-individual variation exists in the severity of toxicity. Genetic variation in the production of the reactive metabolite, N-acetyl-p-benzoquinonimine (NAPQI), accounts for some of that variation. In addition, variable detoxification of NAPQI, accomplished in part by glutathione conjugation, may be important.

### SUMMARY

This document is based in part on the discovery that single nucleotide polymorphisms (SNPs) on chromosome 3 may be useful as biomarkers to predict the severity of acetaminophen toxicity. As described herein, experiments were conducted to identify basal expression and SNPs associated with NAPQI toxicity, and to characterize mRNA expression changes that occur after exposure to NAPQI. These experiments suggested that variation in the basal expression of glutathione pathway genes could explain 37.3% of the NAPQI IC₅₀ variation in this model system. For example, genome-wide association of basal expression with IC₅₀ revealed that the PXR/RXR activation pathway was the most highly associated canonical pathway (p=3.23×10⁻³). Further, a genome-wide SNP analysis identified a group of four linked SNPs on chromosome 3 that were highly associated with NAPQI toxicity (p=7.5×10⁻⁸ for most highly associated SNP). These SNPs are in a highly conserved “gene desert,” but in gel shift assays, binding was observed at the locus of the most highly associated SNP. mRNA expression differences also were observed between the most sensitive and resistant cell lines in terms of extent of change and the pathways altered.

In one aspect, this document features a method for predicting the likelihood of acetaminophen toxicity in a subject, comprising (a) determining whether a biological sample from the subject comprises a wild type or variant rs2880961 allele, and (b) classifying the subject as having a greater likelihood of acetaminophen toxicity if the variant allele is present in the biological sample, and classifying the subject as having a lesser likelihood of acetaminophen toxicity if the wild type allele is present in the biological sample. The subject can be, for example, a human.

In another aspect, this document features a method for determining a tolerable dose of acetaminophen for administration to a subject, comprising (a) determining whether a biological sample from the subject comprises a wild type or variant rs2880961 allele, and (b) determining that the tolerable dose is lower if the variant allele is present in the biological sample, and determining that the tolerable dose is higher if the wild type allele is present in the biological sample. The subject can be, e.g., a human.

In another aspect, this document features a method of assessing likelihood of acetaminophen toxicity in a subject, comprising (a) receiving a biological sample obtained from the subject, (b) assaying the sample to determine whether the sample comprises a wild type or variant rs2880961 allele, (c) communicating to a medical practitioner information about whether the wild type or variant allele is present in the sample, and (d) before or after step (a), communicating to a medical practitioner information indicating that the presence of the variant allele correlates with acetaminophen toxicity. The subject can be, e.g., a human.

In still another aspect, this document features a method for determining a tolerable dose of acetaminophen for administration to a subject, comprising (a) receiving a biological sample obtained from the subject, (b) assaying the sample to determine whether the sample comprises a wild type or variant rs2880961 allele, (c) communicating to a medical practitioner information about whether the wild type or variant allele is present in the sample, and (d) before or after step (a), communicating to a medical practitioner information indicating that the presence of the variant allele correlates with a lower suggested dose. The subject can be, for example, a human.

In another aspect, this document features a method for predicting susceptibility to liver damage in a subject, comprising (a) determining whether a biological sample from the subject comprises a wild type or variant rs2880961 allele, and (b) classifying the subject as having a greater susceptibility to liver damage if the variant allele is present in the biological sample, and classifying the subject as having a lesser susceptibility to liver damage if the wild type allele is present in the biological sample. The subject can be, e.g., a human.

In still another aspect, this document features a method for predicting susceptibility to liver damage in a subject, comprising (a) receiving a biological sample obtained from the subject, (b) assaying the sample to determine whether the sample comprises a wild type or variant rs2880961 allele, (c) communicating to a medical practitioner information about whether the wild type or variant allele is present in the sample, and (d) before or after step (a), communicating to a medical practitioner information indicating that the presence of the variant allele correlates with greater susceptibility to liver damage. The subject can be, for example, a human.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram of the human variation cell line model system described herein.

FIGS. 2A and 2B are plots of IC₅₀ vs. mRNA expression (FIG. 2A) or SNPs (FIG. 2B) based on P-value and chromosomal position. The y-axis of each graph is the −log₁₀(p-value) for the association, while the x-axis gives the chromosome of the probe set or SNP and relative position on the chromosome. Each dot represents an association identified. The red dots in FIG. 2A represent “glutathione pathway” probe sets. Red dots in FIG. 2B represent SNPs in “glutathione pathway” genes.

FIG. 3 is a schematic diagram of the chromosome 3 region that includes SNPs found to be associated with NAPQI IC₅₀, as described herein. The x-axis gives the position on chromosome 3, while the y-axis indicates the −log(p-value) of the SNP-IC₅₀ association. Colored markers represent SNPs in the region of chromosome 3 that is highly associated with NAPQI IC₅₀. Relative positions of transcripts in the EST database (small red bars), potentially novel genes or pseudogenes predicted to be in the region (green bars), and an evolutionarily conserved region with many predicted transcription factor binding sites (black bar) also are shown.

FIG. 4 is a picture of an electrophoretic mobility shift assay (EMSA) showing shifts observed with WT and variant rs2880961 probes in the presence of a pooled lymphoblastoid cell nuclear extract.

### DETAILED DESCRIPTION

### 1. Genotype-Phenotype Association Studies

This document relates to results obtained using a “pharmacogenomic panel” of immortalized human lymphoblastoid cell lines obtained from healthy individuals of varying ethnicities that can be used for preclinical pharmacogenomic testing for common, functionally significant gene sequence variation that influences drug response phenotypes. Pharmaceutical companies could, for example, test drugs on this panel of cell lines prior to testing the drugs on patients. Medical researchers could use the cell line panel to determine genetic reasons for adverse drug reactions, or failure of a drug to be efficacious. A pharmacogenomic panel of cell lines can be used to test any type of therapeutic agent, including, without limitation, anti-cancer drugs (e.g., taxanes such as docetaxel and paclitaxel, cisplatin, anthrcyclines such as doxorubicin and epirubicin, and thiopurines such as 6-mercaptopurine and 6-thioguanine) and immunosuppressants (e.g., mycophenolic acid). A pharmacogenomic cell line panel also can be used to test drug metabolites such as NAPQI, a toxic metabolite of acetaminophen. Further, such a panel can be used to test individual responses to radiation treatment, for example.

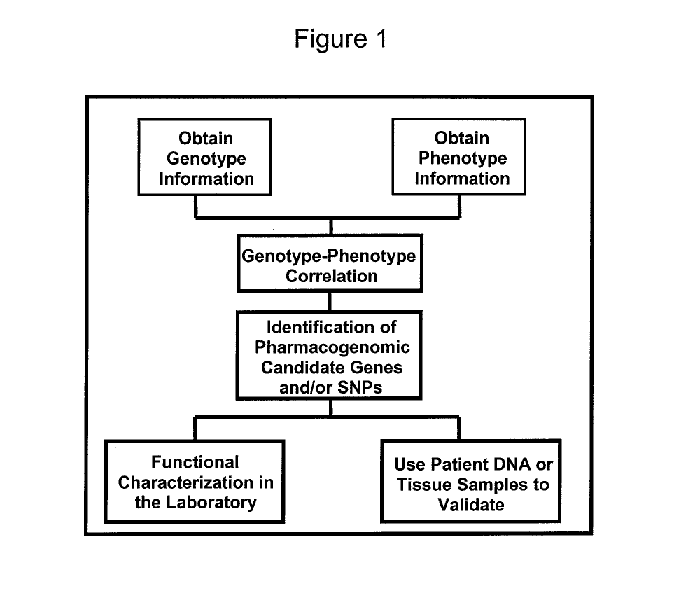

Drug response phenotypes can vary from life-threatening adverse drug reactions at one end of the spectrum to lack of the desired therapeutic efficacy at the other. Thus, a cell line panel such as that described herein can be used to define, prior to patient drug exposure, the possible effect of common DNA sequence variation on drug response. For example, in depth resequencing data can be obtained in the cell lines for genes encoding proteins in known pathways for drug metabolism, drug transport, and drug effects. In addition, genome-wide single nucleotide polymorphisms (SNPs) across the entire genome can be obtained for the individual cell lines for use in genome-wide association studies. Genotype-phenotype correlation analyses using SNPs and intragene haplotype (the combination of SNPs on a given allele) resulting from gene resequencing and genome-wide SNPs can be performed to identify pharmacogenomic candidate genes, both within traditional pharmacokinetic (PK) and pharmacodynamic (PD) pathways, as well as across the entire genome. Expression array data for every gene in the human genome encoding a protein, as well as exon array data and genome-wide gene copy number information also can be obtained for the cell lines. Further, as future techniques for defining DNA sequence variation are developed, culminating in complete genomic sequence for each cell line, those techniques can be added to accumulate a dense array of information—in effect, a “data warehouse”—with respect to differences in DNA sequence and structure that can be correlated with variation in drug-related phenotypes. Those phenotypes may include variation in gene expression, variation in cytotoxicity, variation in apoptosis, variation in nucleic acid methylation, and variation in metabolites in response to varying concentrations of drug. All of this information can be used to perform both “pathway-based” and “genome-wide” genotype-phenotype correlations to identify genetic polymorphisms and/or haplotypes that can be used to develop hypotheses with the cell lines, which then can be tested functionally in the laboratory and also in the clinic, using patient DNA or tissue samples (see FIG. 1). Therefore, the panel of cell lines described herein can be used to identify and characterize the effect of common variation in DNA sequence and structure in human populations on drug response phenotypes that might be responsible for individual differences in adverse drug reactions or clinical drug efficacy. It is noted that in addition to sequence information, data related to levels of metabolites, polypeptides, and mRNAs can be obtained from the panel of cell lines and correlated to individual variation in drug effects.

Cells used in the model system described herein can be obtained commercially, for example, from the non-profit Coriell Institute for Medical Research (online at cimr.umdnj.edu). For example, the Human Variation Panel cell lines available from Coriell can be used. The Human Variation Panel includes immortalized lymphoblastoid cell lines collected from 100 African American (AA), 100 Caucasian American (CA), 100 Han-Chinese American (HCA) subjects and 23 CEPH (Utah family) cell lines. The panel used in the methods described herein can include any suitable number of individual cell lines from any ethnic group. For example, the panel can include from 50 to 100 individual AA cell lines, from 50 to 100 CA cell lines, and/or from 50 to 100 HCA cell lines. DNA from the cell lines can be used for in depth resequencing of genes of interest, and also to obtain genome-wide SNP data for use during genome-wide association studies. The advantage of this system is that the cells are “renewable” and broadly accessible to the general scientific community. In addition, these cell lines represent ethnically diverse population groups.

Modern genomic tools (e.g., genome-wide SNPs and in depth resequencing of functionally important genes) can be used with the cell line panel to identify genes that might be associated with drug response phenotypes. Phenotypes correlated with this genetic variation can include, for example, expression array and metabolomic data, drug-induced cytotoxicity, methylation status, copy number, and cell cycle effects. SNPs or genes showing significant association with these phenotypes then can be tested functionally and, eventually, clinically. In essence, each of the cell lines in the panel can be viewed as an individual “patient” with a unique genotype and a series of associated phenotypes that can be used for preclinical screening of pharmacogenomic candidate genes and SNPs. A tremendous advantage of this model system is the fact that high throughput genetic data for these cell lines can be added continuously. Therefore, unlike patient-based information, data for these cell lines can “accumulate” and be used for studies involving a variety of drug response phenotypes and a virtually endless series of drugs or drug candidates.

SNP and haplotype associations can be performed with cell-based phenotypes and/or with phenotypes related to the response to treatment of disease with particular therapeutics. Cell-based phenotypes include, for example, drug cytotoxicity, levels of intracellular drug metabolites, and gene expression before and after drug treatment in lymphoblastoid cells. Patient-related phenotypes include, for example, overall patient survival and/or time to progression after treatment, as well as drug-related toxicity phenotypes, including neutrophil and platelet counts.

The association of each SNP with the quantitative phenotypes of metabolite concentration, cytotoxicity, and level of gene expression, as well as neutrophil and platelet counts can be evaluated with linear models in which genotypes for a SNP are evaluated with two indicators as covariates. This provides a 2 degree-of-freedom (df) test for each SNP. To assess single SNP genotype associations with patient survival time and time to progression, the Kaplan-Meier method can be used to estimate survival curves for the different genotypes. The curves can be compared using log-rank tests. Survival time as a function of genotype can be examined using the Cox proportional hazards model, and hazard ratios can be used to examine the survival rate by genotype (Cox (1972) Journal of the Royal Statistical Society Series B: 187-220). Disease status, age at time of treatment, gender and duration of treatment can be included as covariates in the proportional hazards models.

In addition to the association of phenotypes with SNPs, their association with intragene haplotypes can be evaluated for candidate genes using a global test of association. Since haplotypes are not observed directly, unknown phase can be accounted for using the score statistics developed by Schaid et al ((2002) Am J Hum Genet. 70:425-34). To estimate the magnitude of effects from haplotypes found to be significant using the score statistics, haplotype regression methods can be used. See, e.g., Lake et al. (2003) Hum Hered 55:56-65. Intragene haplotypes can be associated with gemcitabine clinical response using survival time and time to progression as phenotypes. All possible pairs of haplotypes can be evaluated for each patient, and the posterior probability can be associated with each haplotype using the EM algorithm, as implemented in the Splus library Haplostat (Schaid et al., supra). These posterior probabilities can be used to create expected design matrices to evaluate the association of haplotypes with survival time via the Cox model.

In addition to sequence information, data related to levels of one or more metabolites, polypeptides, and/or RNAs (e.g., mRNAs) can be obtained from cell lines and correlated to drug responses. Cell lines can be characterized for any number of SNPs, metabolites, polypeptides, and RNAs (e.g., at least 100, at least 1,000, at least 10,000, at least 20,000, at least 50,000, or at least 100,000 SNPs, metabolites, polypeptides, or RNAs). In some embodiments, a cell can be characterized for all SNPs, and levels of all metabolites, all polypeptides, and/or all mRNAs.

In some cases, information obtained for particular therapeutic agents can be extrapolated to other agents that have similar metabolic pathways. For example, data obtained for a pyrimidine analog such as gemcitabine can be extrapolated to other pyrimidine analogs such as AraC, 5-fluorouracil (5-FU), and the 5-FU prodrug, capecitabine.

Further, information regarding the cellular response (e.g., apoptosis and metabolism) in various ethnic groups for various doses of particular agents can be obtained to determine whether higher or lower doses may be needed for efficacy and/or to avoid toxicity. For example, if it is determined that a particular ethnicity is likely to be more resistant to a therapeutic agent, a higher dose can be used, whereas if it is determined that a particular ethnicity is likely to be more responsive to the agent, a lower dose may be used. If it is determined that a particular ethnicity is more likely to experience toxicity in response to a therapeutic agent, a lower dose can be used, whereas if it is determined that a particular ethnicity is less likely to experience toxicity in response to the agent, a higher dose may be used.

### 2. Acetaminophen

Acetaminophen (APAP) is widely used for its analgesic and antipyretic activities. Although it is considered by many to be a “safe” drug, as a result of accidental and intentional overdose, it is the leading cause of acute liver failure in the United States (Larson et al. (2005) Hepatology 42:1364-1372). Additionally, APAP can cause elevated aminotransferase levels in healthy adults when administered at the upper limit of the recommended dose (Watkins et al. (2006) JAMA 296:87-93). Although acetaminophen toxicity occurs frequently, success in treating life-threatening overdoses is limited. The mortality rate of patients who present with hepatic failure is reported to range from 20 to 40 percent (Makin et al. (1995) Gastroenterol. 109:1907-1916; and Schiodt et al. (1997) New Engl. J. Med. 337:1112-1117).

Acetaminophen is metabolized primarily by sulfation and glucuronidation when taken in therapeutic doses (Vermeulen et al (1992) Drug Metab. Rev. 24:367-407). However, CYP2E1, CYP1A2, and CYP3A4 convert 5 to 9 percent of acetaminophen to a reactive metabolite, NAPQI (Corcoran et al. (1980) Mol. Pharmacol. 18:536-542; and Dahlin et al. (1984) Proc. Natl. Acad. Sci. USA 81:1327-1331). NAPQI detoxification occurs through glutathione (GSH) conjugation. After GSH depletion, however, it is postulated that the NAPQI can exert hepatotoxic effects by binding to cellular macromolecules, although the exact mechanism of toxicity remains controversial (Mitchell et al. (1973) J. Pharmacol. Exp. Ther. 187:211-217; Coles et al. (1988) Arch. Biochem. Biophys. 264:253-260; and Rogers et al. (1997) Chem. Res. Toxicol. 10:470-476). N-acetylcysteine, which restores hepatic glutathione, can prevent or limit liver injury. Therefore, N-acetylcysteine is currently used in the treatment of acetaminophen overdose (Mitchell et al., supra; and Prescott et al. (1974) Lancet 1:588-592). After hepatic failure has developed, however, N-acetylcysteine administration is associated with only a 21-28% reduction in mortality (Harrison et al. (1990) Lancet 335:1572-1573; and Keays et al. (1991) Brit. Med. J. 303:1026-1029).

The Rumack-Matthew nomogram is used clinically to determine if a patient who presents to a health care facility within 24 hours after a single acute acetaminophen ingestion should be treated with N-acetylcysteine, by predicting the likelihood of hepatotoxicity based on plasma APAP concentration (Rumack and Matthew (1975) Pediatrics 55:871-876). This nomogram is not useful if the time of ingestion is unknown or if toxicity is suspected to result from repeated supratherapeutic doses (Heard (2008) New Engl. J. Med. 359:285-292). Genetic variation in drug metabolism and susceptibility to liver injury may be important factors to consider in addition to plasma acetaminophen concentration when making treatment decisions. Therefore, identification of genetic biomarkers that contribute to variation in APAP toxicity could be useful in developing treatment algorithms, particularly in cases in which the Rumack-Matthew nomogram is not useful.

As described herein, experiments were conducted to characterize genetic variation that may contribute to differences in toxicity after exposure to NAPQI, using a “Human Variation Panel” lymphoblastoid cell line-based model system. The associations between NAPQI-induced cytotoxicity and both single nucleotide polymorphisms (SNPs) and basal mRNA expression in cell lines can be evaluated to identify biomarkers that can be used to predict the severity of APAP toxicity and to individualize therapy for APAP overdose. Utilizing NAPQI rather than APAP can allow for evaluation of the variation in toxicity of the active metabolite of acetaminophen, rather than the parent drug, which can be variably converted into NAPQI. For example, NAPQI IC₅₀ values, genome-wide SNPs, and genome-wide basal expression array data can be obtained for a population of lymphoblastoid cell lines. After adjustment (e.g., for race, gender, age, or any other suitable factor), genotype-phenotype association analyses can be performed for NAPQI IC₅₀, basal expression, and SNP genotypes. Sequence variation in the glutathione pathway (which is responsible for detoxification of NAPQI), as well as genome-wide expression and SNPs, can be evaluated for association with IC₅₀. In addition, pre- and post-NAPQI mRNA expression patterns can be compared in sensitive and resistant lymphoblastoid cells, as well as HepG2 cells. The use of such a model system can allow for identification of novel SNPs and mRNA transcripts that may be useful biomarkers to test in clinical studies of acetaminophen overdose.

### 3. Methods

This document provides methods for assessing a subject's likelihood of acetaminophen toxicity, and/or for determining acetaminophen dose levels. The methods provided herein can include, for example, testing a biological sample obtained from a subject to determine whether the sample contains a biomarker indicating that the subject is likely to experience acetaminophen toxicity. Such methods also can be used to, for example, determine whether a subject should be treated with a lower rather than a higher dose of acetaminophen (e.g., if a biological sample from the subject contains a nucleotide polymorphism associated with acetaminophen toxicity, it can be an indication that the subject should be treated with a lower dose of acetaminophen than if the subject did not contain the polymorphism).

Any suitable biological sample can be used. A biological sample can be, for example, blood, serum, plasma, urine, cerebrospinal fluid, pleural fluid, sputum, peritoneal fluid, bladder washings, oral washings, tissue samples, touch preps, or fine-needle aspirates.

In some embodiments, a biomarker can be a nucleotide sequence variant (e.g., rs2880961). Nucleotide sequence variants can be detected, for example, by sequencing exons, introns, 5′ untranslated sequences, or 3′ untranslated sequences, by performing allele-specific hybridization, allele-specific restriction digests, mutation specific polymerase chain reactions (MSPCR), by single-stranded conformational polymorphism (SSCP) detection (Schafer et al. (1995) Nat. Biotechnol. 15:33-39), denaturing high performance liquid chromatography (DHPLC, Underhill et al. (1997) Genome Res. 7:996-1005), infrared matrix-assisted laser desorption/ionization (IR-MALDI) mass spectrometry (WO 99/57318), and combinations of such methods.

Genomic DNA generally is used in the analysis of nucleotide sequence variants, although mRNA also can be used. Genomic DNA is typically extracted from a biological sample such as a peripheral blood sample, but can be extracted from other biological samples, including tissues (e.g., mucosal scrapings of the lining of the mouth or from renal or hepatic tissue). Routine methods can be used to extract genomic DNA from a blood or tissue sample, including, for example, phenol extraction. Alternatively, genomic DNA can be extracted with kits such as the QIAamp® Tissue Kit (Qiagen, Chatsworth, Calif.) and the Wizard® Genomic DNA purification kit (Promega).

An amplification step typically is performed before proceeding with the detection method. For example, exons or introns of a gene can be amplified and then directly sequenced. Dye primer sequencing can be used to increase the accuracy of detecting heterozygous samples.

Allele specific hybridization is an example of a method that can be used to detect sequence variants, including complete haplotypes of a subject (e.g., a mammal such as a human). See, Stoneking et al (1991) Am. J. Hum. Genet. 48:370-382; and Prince et al. (2001) Genome Res. 11: 152-162. In practice, samples of DNA or RNA from one or more mammals can be amplified using pairs of primers and the resulting amplification products can be immobilized on a substrate (e.g., in discrete regions). Hybridization conditions are selected such that a nucleic acid probe can specifically bind to the sequence of interest, e.g., the variant nucleic acid sequence. Such hybridizations typically are performed under high stringency as some sequence variants include only a single nucleotide difference. High stringency conditions can include the use of low ionic strength solutions and high temperatures for washing. For example, nucleic acid molecules can be hybridized at 42° C. in 2×SSC (0.3M NaCl/0.03 M sodium citrate/0.1% sodium dodecyl sulfate (SDS) and washed in 0.1×SSC (0.015M NaCl/0.0015 M sodium citrate), 0.1% SDS at 65° C. Hybridization conditions can be adjusted to account for unique features of the nucleic acid molecule, including length and sequence composition. Probes can be labeled (e.g., fluorescently) to facilitate detection. In some embodiments, one of the primers used in the amplification reaction is biotinylated (e.g., 5′ end of reverse primer) and the resulting biotinylated amplification product is immobilized on an avidin or streptavidin coated substrate.

Allele-specific restriction digests can be performed in the following manner. For nucleotide sequence variants that introduce a restriction site, restriction digest with the particular restriction enzyme can differentiate the alleles. For sequence variants that do not alter a common restriction site, mutagenic primers can be designed that introduce a restriction site when the variant allele is present or when the wild type allele is present. A portion of a nucleic acid can be amplified using the mutagenic primer and a wild type primer, followed by digest with the appropriate restriction endonuclease.

Certain variants, such as insertions or deletions of one or more nucleotides, change the size of the DNA fragment encompassing the variant. The insertion or deletion of nucleotides can be assessed by amplifying the region encompassing the variant and determining the size of the amplified products in comparison with size standards. For example, a region of a gene can be amplified using a primer set from either side of the variant. One of the primers is typically labeled, for example, with a fluorescent moiety, to facilitate sizing. The amplified products can be electrophoresed through acrylamide gels with a set of size standards that are labeled with a fluorescent moiety that differs from the primer.

PCR conditions and primers can be developed that amplify a product only when the variant allele is present or only when the wild type allele is present (MSPCR or allele-specific PCR). For example, patient DNA and a control can be amplified separately using either a wild type primer or a primer specific for the variant allele. Each set of reactions is then examined for the presence of amplification products using standard methods to visualize the DNA. For example, the reactions can be electrophoresed through an agarose gel and the DNA visualized by staining with ethidium bromide or other DNA intercalating dye. In DNA samples from heterozygous patients, reaction products would be detected in each reaction. Patient samples containing solely the wild type allele would have amplification products only in the reaction using the wild type primer. Similarly, patient samples containing solely the variant allele would have amplification products only in the reaction using the variant primer. Allele-specific PCR also can be performed using allele-specific primers that introduce priming sites for two universal energy-transfer-labeled primers (e.g., one primer labeled with a green dye such as fluorescein and one primer labeled with a red dye such as sulforhodamine). Amplification products can be analyzed for green and red fluorescence in a plate reader. See, Myakishev et al. (2001) Genome 11:163-169.

Mismatch cleavage methods also can be used to detect differing sequences by PCR amplification, followed by hybridization with the wild type sequence and cleavage at points of mismatch. Chemical reagents, such as carbodiimide or hydroxylamine and osmium tetroxide can be used to modify mismatched nucleotides to facilitate cleavage.

In some embodiments, a biomarker can be a variant polypeptide. Alternatively, antibodies having specific binding affinity can be used to detect variant polypeptides. Variant polypeptides can be produced in various ways, including recombinantly, as known in the art. Host animals such as rabbits, chickens, mice, guinea pigs, and rats can be immunized by injection of a variant polypeptide. Various adjuvants that can be used to increase the immunological response depend on the host species and include Freund's adjuvant (complete and incomplete), mineral gels such as aluminum hydroxide, surface active substances such as lysolecithin, pluronic polyols, polyanions, peptides, oil emulsions, keyhole limpet hemocyanin, and dinitrophenol. Polyclonal antibodies are heterogeneous populations of antibody molecules that are contained in the sera of the immunized animals. Monoclonal antibodies, which are homogeneous populations of antibodies to a particular antigen, can be prepared using a variant polypeptide and standard hybridoma technology. In particular, monoclonal antibodies can be obtained by any technique that provides for the production of antibody molecules by continuous cell lines in culture such as described by Kohler et al. (1975) Nature 256:495, the human B-cell hybridoma technique (Kosbor et al. (1983) Immunology Today 4:72; Cote et al. (1983) Proc. Natl. Acad. Sci. USA 80:2026), and the EBV-hybridoma technique (Cole et al., Monoclonal Antibodies and Cancer Therapy, Alan R. Liss, Inc., pp. 77-96 (1983). Such antibodies can be of any immunoglobulin class including IgG, IgM, IgE, IgA, IgD and any subclass thereof. A hybridoma producing monoclonal antibodies can be cultivated in vitro or in vivo.

Antibody fragments that have specific binding affinity for a variant polypeptide can be generated using known techniques. For example, such fragments include but are not limited to F(ab′)2 fragments that can be produced by pepsin digestion of the antibody molecule, and Fab fragments that can be generated by reducing the disulfide bridges of F(ab′)2 fragments. Alternatively, Fab expression libraries can be constructed. See, for example, Huse et al. (1989) Science 246:1275. Once produced, antibodies or fragments thereof are tested for recognition of GSTO2 variant polypeptides by standard immunoassay methods including ELISA techniques, radioimmunoassays and Western blotting. See, Short Protocols in Molecular Biology, Chapter 11, Green Publishing Associates and John Wiley & Sons, edited by Ausubel et al., 1992.

In some embodiments, a biomarker can be a level of a nucleic acid (e.g., an RNA) polypeptide, or metabolite that is altered with respect to, for example, a control level of the nucleic acid, polypeptide, or metabolite. Levels of nucleic acids, polypeptides, and metabolites can be determined using any suitable methods, including those that are known in the art. These include, for example, antibody-based methods, reverse transcriptase PCR (RT-PCR) methods, and any other methods that can be used to measure the level of a nucleic acid, polypeptide, or metabolite in a biological sample.

As discussed herein, the methods provided herein can be used to predict the likelihood of acetaminophen toxicity in a subject (e.g., a mammal such as a rat, a dog, or a human). For example, a method can include determining whether a biological sample from a subject comprises a wild type or variant rs2880961 allele, and classifying the subject as having a greater likelihood of acetaminophen toxicity if the variant allele is present in the biological sample, and classifying the subject as having a lesser likelihood of acetaminophen toxicity if the wild type allele is present in the biological sample. Such methods also can be used to determine a tolerable dose of acetaminophen for administration to a subject. For example, using a method as described herein, it can be determined that a tolerable dose for a particular subject is lower if the variant rs2880961 allele is present in a biological sample from the subject, and determining that the tolerable dose is higher if the wild type rs2880961 allele is present in the biological sample.

In some embodiments, a method for assessing likelihood of acetaminophen toxicity in a subject can include receiving a biological sample obtained from the subject, assaying the sample to determine whether it contains a wild type or variant rs2880961 allele, communicating to a medical practitioner information about whether the wild type or variant allele is present in the sample, and, before or after the first step, communicating to a medical practitioner information indicating that the presence of the variant allele correlates with acetaminophen toxicity. Similarly, a method for determining a tolerable dose of acetaminophen for administration to a subject can include receiving a biological sample obtained from a subject, assaying the sample to determine whether it contains a wild type or variant rs2880961 allele, communicating to a medical practitioner information about whether the wild type or variant allele is present in the sample, and, before or after the first step, communicating to a medical practitioner information indicating that the presence of the variant allele correlates with a lower suggested dose.

This document also provides methods for predicting susceptibility to liver damage in a subject. The methods provided herein can include, for example, testing a biological sample obtained from a subject to determine whether the sample contains a biomarker (e.g., a wild type or variant rs2880961 allele) indicating the susceptibility of the subject to liver toxicity. In some embodiments, the subject can be classified as having greater susceptibility to liver damage if the sample contains a particular biomarker (e.g., a variant rs2880961 allele), or having lesser susceptibility to liver damage if the sample does not contain the biomarker or contains a different biomarker (e.g., a wild type rs2880961 allele). Such methods also can be used to, for example, determine whether a subject should be treated with a lower rather than a higher dose of a compound or medicament (e.g., if a biological sample from the subject contains a nucleotide polymorphism associated with greater susceptibility to liver toxicity, it can be an indication that the subject should be treated with a lower dose of the compound or medicament than if the subject did not contain the polymorphism).

As discussed herein, the methods provided herein can be used to predict a subject's susceptibility to liver damage in a subject (e.g., a mammal such as a rat, a dog, or a human). For example, a method can include determining whether a biological sample from a subject comprises a wild type or variant rs2880961 allele, and classifying the subject as having a greater susceptibility to liver toxicity if the variant allele is present in the biological sample, and classifying the subject as having a lesser susceptibility to liver toxicity if the wild type allele is present in the biological sample. Such methods also can be used to determine a tolerable dose of a compound or medicament for administration to a subject. For example, using a method as described herein, it can be determined that a tolerable dose of a medicament for a particular subject is lower if the variant rs2880961 allele is present in a biological sample from the subject, and determining that the tolerable dose of the medicament is higher if the wild type rs2880961 allele is present in the biological sample.

In some embodiments, a method for predicting susceptibility to liver damage in a subject can include receiving a biological sample obtained from the subject, assaying the sample to determine whether it contains a wild type or variant rs2880961 allele, communicating to a medical practitioner information about whether the wild type or variant allele is present in the sample, and, before or after the first step, communicating to a medical practitioner information indicating that the presence of the variant allele correlates with greater susceptibility to liver toxicity.

This document also provides methods and materials to assist medical or research professionals in determining whether or not a subject is likely to experience acetaminophen toxicity, to determine a tolerable dose of acetaminophen, or predict susceptibility of a subject to liver damage. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining whether a wild type or variant rs2880961 allele is present in a biological sample from a subject, and (2) communicating information about the allele to that professional.

After information about the rs2880961 allele is reported, a medical professional can take one or more actions that can affect patient care. For example, a medical professional can record information in the patient's medical record regarding the likelihood of the patient to experience acetaminophen toxicity, a tolerable dose of acetaminophen for the patient, or the likelihood of liver damage in the patient. In some cases, a medical professional can record a diagnosis acetaminophen toxicity, or otherwise transform the patient's medical record, to reflect the patient's medical condition. In some cases, a medical professional can review and evaluate a patient's entire medical record, and assess multiple treatment strategies, for clinical intervention of a patient's condition.

A medical professional can initiate or modify treatment after receiving information regarding a patient's likelihood of experiencing acetaminophen toxicity, for example. In some cases, a medical professional can recommend a change in therapy. In some cases, a medical professional can enroll a patient in a clinical trial for acetaminophen alternatives, for example.

A medical professional can communicate information regarding the likelihood of acetaminophen toxicity, liver damage, or tolerable acetaminophen doses to a patient or a patient's family. In some cases, a medical professional can provide a patient and/or a patient's family with information regarding acetaminophen toxicity and liver damage, including treatment options, prognosis, and referrals to specialists. In some cases, a medical professional can provide a copy of a patient's medical records to a specialist.

A research professional can apply information regarding a subject's likelihood of experiencing acetaminophen toxicity or liver damage advance scientific research. For example, a researcher can compile data on wild type and variant rs2880961 alleles, as well as information regarding acetaminophen toxicity, tolerable acetaminophen dosages, and susceptibility to liver damage. In some cases, a research professional can obtain a subject's rs2880961 haplotype levels to evaluate a subject's enrollment, or continued participation in a research study or clinical trial. In some cases, a research professional can communicate information regarding a subject's likelihood of experiencing acetaminophen toxicity, tolerable acetaminophen dosages, and susceptibility to liver damage, to a medical professional. In some cases, a research professional can refer a subject to a medical professional.

Any appropriate method can be used to communicate information to another person (e.g., a professional). For example, information can be given directly or indirectly to a professional. For example, a laboratory technician can input rs2880961 allele into a computer-based record. In some cases, information is communicated by making an physical alteration to medical or research records. For example, a medical professional can make a permanent notation or flag a medical record for communicating a diagnosis to other medical professionals reviewing the record. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

This document also provides articles of manufacture that can include, for example, materials and reagents that can be used to determine whether a subject has a biomarker for acetaminophen toxicity. An article of manufacture can include, for example, nucleic acids and/or polypeptides immobilized on a substrate (e.g., in discrete regions, with different populations of isolated nucleic acids or polypeptides immobilized in each discrete region). Suitable substrates can be of any shape or form and can be constructed from, for example, glass, silicon, metal, plastic, cellulose, or a composite. For example, a suitable substrate can include a multiwell plate or membrane, a glass slide, a chip, or polystyrene or magnetic beads. Nucleic acid molecules or polypeptides can be synthesized in situ, immobilized directly on the substrate, or immobilized via a linker, including by covalent, ionic, or physical linkage. Linkers for immobilizing nucleic acids and polypeptides, including reversible or cleavable linkers, are known in the art. See, for example, U.S. Pat. No. 5,451,683 and WO98/20019. Immobilized nucleic acid molecules are typically about 20 nucleotides in length, but can vary from about 10 nucleotides to about 1000 nucleotides in length.

In practice, to detect a particular allele of a nucleic acid, for example, a sample of DNA or RNA from a subject can be amplified, the amplification product hybridized to an article of manufacture containing populations of isolated nucleic acid molecules in discrete regions, and hybridization can be detected. Typically, the amplified product is labeled to facilitate detection of hybridization. See, for example, Hacia et al. (1996) Nature Genet. 14:441-447; and U.S. Pat. Nos. 5,770,722 and 5,733,729.

The invention will be further described in the following examples, which do not limit the scope of the invention described in the claims.

### EXAMPLES

### Example 1

### Methods and Materials

Cell Lines Lymphoblastoid cells derived from 60 Caucasian-American (CA) subjects, 56 African-American (AA) subjects, and 60 Han Chinese American (HCA) subjects were obtained from the Coriell Cell Repository (Camden, N.J.). The National Institute of General Medical Sciences had anonymized these cell lines before deposit, and all subjects had provided written informed consent for the use of their samples for research purposes. HepG2 cells were purchased from the American Type Culture Collection (ATCC; Manassas, Va.).

NAPQI cytotoxicity experiments: NAPQI was purchased from Dalton Pharma Services (Toronto, ON, Canada) and was dissolved in DMSO immediately prior to use. After plating each lymphoblastoid cell line at a concentration of 5×10⁴ cells/well, NAPQI dissolved in 1% DMSO was applied to each cell line for 24 hours at 7 concentrations ranging from 0 to 100 μM. The cytotoxic effect of NAPQI was evaluated by determining the concentration required to inhibit cell growth by 50% (IC₅₀). Specifically, after incubation with NAPQI for 24 hours, the CellTiter Blue (Promega, Madison, Wis.) assay was utilized according to the manufacturer's instructions. All experiments were performed in triplicate and IC₅₀ values reported are averages of those three determinations.

mRNA microarray analysis: Total RNA was extracted from lymphoblastoid cell lines and HepG2 cells at baseline or after treatment with the IC₅₀ value for NAPQI specific to that cell line using the RNeasy kit (Qiagen, Valencia, Calif.). RNA quality assessment was performed using the Agilent 2100 bioanalyzer (Agilent Technologies, Inc., Santa Clara, Calif.) prior to microarray analysis. All RNA samples had Agilent RNA Integrity Number (RIN) values greater than 9.0. The RNA was then reverse-transcribed and biotin labeled for hybridization with Affymetrix U133 Plus 2.0 GeneChips (Affymetrix, Santa Clara, Calif.). The microarray images were analyzed using quality control techniques established in the Mayo Clinic Microarray Core Facility.

SNP genotyping: DNA corresponding to each cell line was obtained from the Coriell Cell Repository. SNPs were genotyped for each lymphoblastoid cell line using the Illumina 550k INFINIUM® HumanHap SNP Chip (Illumina Inc., San Diego, Calif.). SNPs for 5 glutathione pathway genes—GSTT1, GSTM1, GSTP1, GSTO1, and GSTO2—were obtained previously by in-depth gene resequencing using DNA from these same cell lines (Moyer et al. (2007) Clin. Cancer. Res. 13:7207-7216; Moyer et al. (2008) Cancer Res. 68:4791-4801; and Mukherjee et al. (2006) Drug Metab. Dispos. 34:1237-1246).

Electrophoretic Mobility Shift Assay (EMSA): Biotin-labeled double-stranded oligonucleotides corresponding to the wild type (WT) sequences and to the variant sequences at rs2880961, together with their corresponding unlabeled oligonucleotides as competitors, were used in these assays. Binding assays were performed, followed by electrophoresis on a 4% nondenaturing gel and transfer to a nylon membrane, with detection according to the manufacturer's directions using the LightShift Chemiluminescent EMSA Kit (Pierce, Rockford, Ill.). Nuclear extracts were prepared from a pool of the lymphoblastoid cell lines used to perform the microarray analyses.

Clinical validation study: DNA was obtained from healthy men and women between 18-45 years of age who had been treated with acetaminophen or placebo and who were not receiving concomitant medications. Subjects received 1000 mg of acetaminophen or placebo orally every six hours for 8 consecutive days. Blood samples were drawn daily prior to dosing. The blood samples were analyzed for alanine aminotransferase (ALT). Baseline ALT was determined as the mean of the values obtained prior to acetaminophen administration. Dosing was discontinued for subjects in whom serum ALT or aspartate aminotransferase (AST) were elevated more than 3 times the upper limit of normal.

Data analysis: Cytotoxicity data were fitted to dose-response curves using the R package, and IC₅₀ values were estimated using the Cedergreen-Ritz-Streibig 5-paramater model. This model, which is a modification of the four-parameter logistic curve, was utilized to take the hormesis observed after application of NAPQI into account. IC₅₀ values were log transformed and adjusted for gender and race.

mRNA expression data were normalized on a log₂ scale using GCRMA. The normalized data were regressed on gender and race. Pearson correlation coefficients were calculated for IC₅₀ and expression levels and the test statistic, given by

$$t = ( \frac{r}{\sqrt{1 - {r}^{2}}} ) \sqrt{n - 2} \sim t ( df = n - 2 )$$

was used to test for a non-zero correlation. These analyses were completed on standardized residuals adjusted for gender and race. The percentage of variation in IC₅₀ explained by glutathione pathway variation was calculated based on the coefficient of determination, r², using a multiple regression model between IC₅₀ values and expression of individual probe sets.

Eigen analysis of SNP data was performed within each race. Genotypes were standardized within each race and principal components analysis was completed within each race. The top 5 principal components for each race were saved and used along with race to adjust genotype data. SNPs were excluded if the call rate was less than 95%, if the minor allele frequency was less than 5%, or if the SNP was out of Hardy-Weinberg equilibrium (p<0.001). One sample corresponding to an African-American subject was removed from the analysis because the SNP genotype call rate across the sample was less than 95%. After quality control, a total of 515,039 SNPs were analyzed. The association between adjusted IC₅₀ and adjusted genotypes were computed. The p-values calculated were based on the F-distribution. Pathway analyses were performed using Ingenuity Pathway Analysis Software (Ingenuity Systems, Redwood City, Calif.).

For the clinical validation study, samples from 70 patients were analyzed. Of the 70 patients, 56 were treated with acetaminophen and 14 were treated with placebo. The effect of treatment (acetaminophen or placebo), genotype (rs2880961), race (Asian, African American, Caucasian, Hispanic, or Caucasian/Hispanic), gender, period (screening/pre-treatment, treatment, recovery), and time within period on ALT was modeled as a mixed model with REML estimated error variance correlation within subject modeled using an AR(1) correlation structure. ALT was transformed to the log scale. All models involving only clinical effects were fully specified, with all interactions considered. For the final models involving the genetic marker, however, all effects except gender were modeled as fully specified. Since gender appeared to have an effect only on overall ALT values, it was modeled without any interactions.

### Example 2

### NAPQI Cytotoxicity

NAPQI cytotoxicity studies were performed to determine the extent of inter-individual variation in IC₅₀ values, as well as to generate a phenotype for association studies to identify potential biomarkers for prediction of risk for toxicity. The average NAPQI IC₅₀ for these cell lines was 6.5±4.5 μM (mean±SD). There were no differences observed in NAPQI IC₅₀ between males and females, p=0.63, or among ethnic groups, p=0.24. At low concentrations of NAPQI, a slight increase in proliferation, suggestive of hormesis, was observed in many of the cell lines.

### Example 3

### Glutathione Pathway Analyses

Variation in the basal expression of glutathione pathway genes could explain 37.3% of the variation in NAPQI IC₅₀ in this “Human Variation Panel” model system. 41 SNPs in 15 genes and 5 mRNA expression probe sets for 4 genes in the glutathione pathway were identified as being associated with IC₅₀, with p<0.05 (Tables 1 and 2). However, none of these associations remained significant after the Bonferroni correction for multiple comparisons.

rs3828599 (p<0.001) and rs8177426 (p=0.001), SNPs in glutathione peroxidase 3, GPX3, were the two SNPs most highly associated with NAPQI IC50 (Table 1). Treatment of HepG2 cells with NAPQI increased the expression of GPX3 1.87 fold (p<0.01) for probe set 214091_s_at and 1.7 fold (p=0.01) for probe set 201348_at. The two “glutathione pathway” probe sets that were most highly associated with NAPQI toxicity both corresponded to GSTA4-202967_at (p=0.001) and 235405_at (p=0.006) (Table 2).

TABLE 1
“Glutathione Pathway” SNPs associated
with NAPQI IC₅₀ with p < 0.05

| SNP | Gene in which SNP is in or near | Raw p value (unadjusted) |
|---|---|---|
| rs3828599 | GPX3 | <0.001 |
| rs8177426 | GPX3 | 0.001 |
| rs7329514 | ABCC4 | 0.002 |
| rs1356553 | GCLC | 0.002 |
| rs17310467 | GSS | 0.003 |
| rs1377392 | GCLC | 0.003 |
| rs1189439 | ABCC4 | 0.003 |
| rs1189437 | ABCC4 | 0.004 |
| rs4773861 | ABCC4 | 0.004 |
| rs10508010 | ABCC4 | 0.005 |
| rs1751043 | ABCC4 | 0.006 |
| rs215063 | ABCC1 | 0.008 |
| rs707148 | GPX3 | 0.008 |
| rs1925860 | ABCC4 | 0.008 |
| rs1189434 | ABCC4 | 0.008 |
| rs3957358 | GCLC | 0.011 |
| E4p254 | GSTM1 | 0.013 |
| rs2074451 | GPX4 | 0.014 |
| rs1766908 | ABCC4 | 0.015 |
| rs215052 | ABCC1 | 0.016 |
| rs2748991 | GSTA2 | 0.017 |
| rs1925856 | ABCC4 | 0.018 |
| rs766606 | ABCC4 | 0.019 |
| rs4715359 | GSTA3 | 0.019 |
| rs2180312 | GSTA2 | 0.020 |
| rs2397105 | GSTA1 | 0.021 |
| rs1925857 | ABCC4 | 0.021 |
| rs12584534 | ABCC4 | 0.025 |
| rs4148530 | ABCC4 | 0.026 |
| rs8191438 | GSTP1 | 0.027 |
| rs1764425 | ABCC4 | 0.030 |
| rs6922172 | GCLC | 0.030 |
| rs8190898 | GSR | 0.032 |
| rs1989983 | ABCC3 | 0.032 |
| rs6060124 | GSS | 0.034 |
| rs8191439 | GSTP1 | 0.034 |
| I6m18 | GSTP1 | 0.040 |
| rs215066 | ABCC1 | 0.045 |
| rs1925851 | ABCC4 | 0.045 |
| rs1029328 | GPX6 | 0.046 |
| rs9474334 | GSTA5 | 0.050 |

TABLE 2
Association between basal expression of “glutathione pathway”
mRNA expression probes and NAPQI IC₅₀, with p < 0.05
Basal Expression vs. NAPQI IC₅₀

| Affymetrix U133 | | | |
|---|---|---|---|
| Plus 2.0 | | | |
| Probeset ID | Gene | p^{†} | r^{§} |
| 202967_at | GSTA4 | 0.001 | −0.24 |
| 235405_at | GSTA4 | 0.006 | −0.21 |
| 222102_at | GSTA3 | 0.020 | −0.17 |
| 211630_s_at | GSS | 0.034 | −0.16 |
| 205439_at | GSTT2 | 0.046 | −0.15 |

^{†}raw p-value;
^{§}Pearson's correlation coefficient

Due to the high homology of GSTA family members, these probe sets may in reality represent a collection of GSTA family members rather than specifically GSTA4. The GSTA family also appeared in the SNP analysis with several SNPs associated with IC₅₀-rs2748991 in GSTA2 (p=0.017), rs4715359 in GSTA3 (p=0.019), rs2180312 in GSTA2 (p=0.020), rs2397105 in GSTA1 (p=0.021), and rs9474334 in GSTA5 (p=0.050). In addition, several GSTA family members were upregulated in HepG2 cells after exposure to NAPQI-GSTA1 was 2.56-fold upregulated (probe set 203924_at, p<0.01), and GSTA4 was upregulated 1.7-fold (202967_at, p<0.01).

### Example 4

### Genome-Wide Expression and SNP Analyses

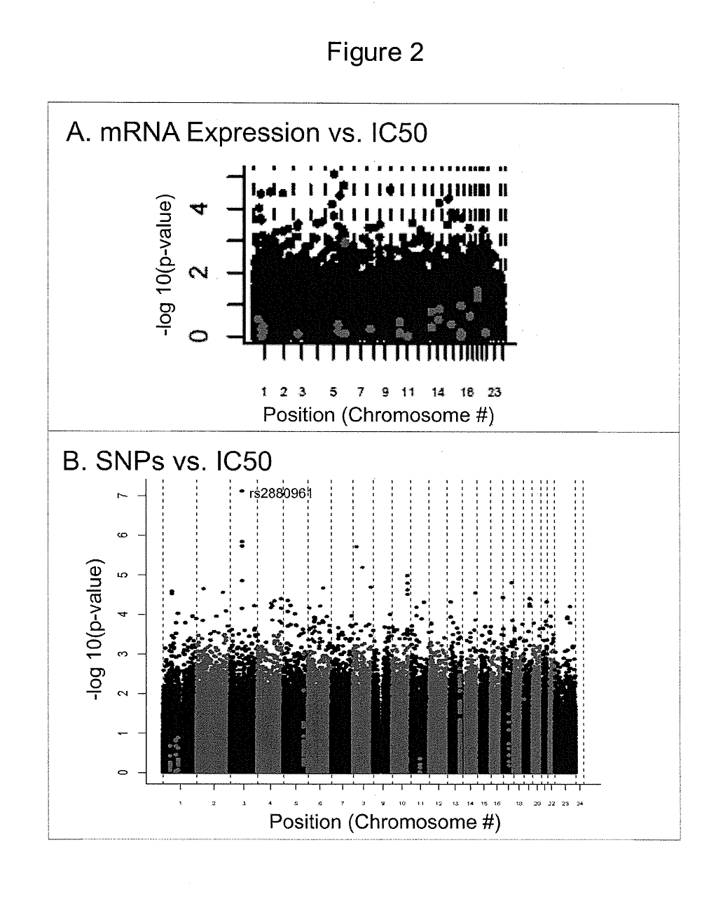

Correlations between basal gene expression or SNP genotypes and NAPQI IC₅₀ values were determined to identify genes that might serve as biomarkers useful for predicting risk for the severity of toxicity. Nineteen expression array probe sets were observed with p-values<0.0001 (FIG. 2A and Table 3), while six would be expected under the null hypothesis. Several individual SNPs had p-values that were much lower than the p-values for probe sets (FIG. 2B and Table 4). When the top hits from the expression analysis (p<1×10⁻³) were compared with the top hits from the SNP analysis (p<1×10⁻³), one gene was identified for which both basal expression and SNP within the gene were associated with IC₅₀. That gene, VAV3 [probe set 218807_at (p=6×10⁻⁴) and rs12071280 (p=9×10⁻⁵)] is a guanine nucleotide exchange factor and a known human oncogene. Additional SNPs may also be associated with IC₅₀ but may regulate mRNA expression of probes that are associated with IC₅₀ through trans-effects. However, those SNPs would not be readily apparent when comparing the results of the SNP-IC₅₀ and expression-IC₅₀ studies.

TABLE 3
Probe sets associated with NAPQI IC₅₀ with p < 0.0001

| Affymetrix | Gene | | q- | corrected | Pearson |
|---|---|---|---|---|---|
| Probe Set ID | Symbol | p-value | value* | p^{†} | r^{§} |
| 226989_at | RGMB | 8.00E−06 | 0.25 | 0.44 | −0.33 |
| 229016_s_at | TRERF1 | 1.77E−05 | 0.25 | 0.97 | −0.32 |
| 206037_at | CCBL1 | 2.46E−05 | 0.25 | 1.00 | −0.31 |
| 229017_s_at | RIPK5 | 2.97E−05 | 0.25 | 1.00 | −0.31 |
| 212698_s_at | SEPT10 | 3.27E−05 | 0.25 | 1.00 | −0.31 |
| 202741_at | PRKACB | 3.43E−05 | 0.25 | 1.00 | 0.31 |
| 227339_at | RGMB | 3.64E−05 | 0.25 | 1.00 | −0.31 |
| 205270_s_at | LCP2 | 3.73E−05 | 0.25 | 1.00 | −0.31 |
| 225792_at | HOOK1 | 4.17E−05 | 0.25 | 1.00 | 0.30 |
| 205204_at | NMB | 4.64E−05 | 0.25 | 1.00 | −0.30 |
| 244063_at | BTN2A1 | 6.09E−05 | 0.27 | 1.00 | −0.30 |
| 205965_at | BATF | 6.22E−05 | 0.27 | 1.00 | −0.30 |
| 223835_x_at | OTP | 7.18E−05 | 0.27 | 1.00 | −0.29 |
| 228583_at | LIN52 | 7.70E−05 | 0.27 | 1.00 | 0.29 |
| 212270_x_at | RPL17 | 7.87E−05 | 0.27 | 1.00 | −0.29 |
| 227370_at | KIAA1946 | 8.00E−05 | 0.27 | 1.00 | −0.29 |
| 214472_at | HIST1H3D | 8.91E−05 | 0.28 | 1.00 | 0.29 |
| 219976_at | HOOK1 | 9.46E−05 | 0.28 | 1.00 | 0.29 |
| 241813_at | MBD1 | 9.82E−05 | 0.28 | 1.00 | −0.29 |

*false discovery rate,
^{†}Bonferroni corrected p-value,
^{§}Pearson's correlation coefficient.

TABLE 4
SNPs associated with NAPQI IC50, p < 0.0001

| | Gene | | | | | | | |
|---|---|---|---|---|---|---|---|---|
| SNP ID | Symbol | Chrom. | Position | MAF | p-value | q-value* | corrected p^{†} | Pearson's r^{§} |
| rs2880961 | C3orf38 | 3 | 88606865 | 0.33 | 7.53E−08 | 0.04 | 0.04 | 0.41 |
| rs2344953 | C3orf38 | 3 | 88614905 | 0.26 | 1.42E−06 | 0.25 | 0.73 | 0.37 |
| rs13101122 | C3orf38 | 3 | 88590539 | 0.48 | 1.86E−06 | 0.25 | 0.96 | 0.37 |
| rs7828851 | CDCA2 | 8 | 25575129 | 0.17 | 1.91E−06 | 0.25 | 0.98 | −0.37 |
| rs4585742 | CPA6 | 8 | 68897323 | 0.23 | 6.34E−06 | 0.65 | 1.00 | −0.35 |
| rs1360864 | ADRA2A | 10 | 112975415 | 0.42 | 1.02E−05 | 0.78 | 1.00 | −0.34 |
| rs6795028 | C3orf38 | 3 | 88563780 | 0.51 | 1.38E−05 | 0.78 | 1.00 | 0.34 |
| rs17767358 | SOX9 | 17 | 67216898 | 0.32 | 1.53E−05 | 0.78 | 1.00 | −0.34 |
| rs7896901 | ADRA2A | 10 | 113000349 | 0.45 | 1.58E−05 | 0.78 | 1.00 | −0.34 |
| rs4508142 | ADRA2A | 10 | 113001028 | 0.45 | 1.59E−05 | 0.78 | 1.00 | −0.34 |
| rs4562278 | MYC | 8 | 128733772 | 0.22 | 2.00E−05 | 0.78 | 1.00 | −0.33 |
| rs11153350 | LAMA4 | 6 | 112685077 | 0.15 | 2.11E−05 | 0.78 | 1.00 | −0.33 |
| rs6715107 | FSHR | 2 | 48996494 | 0.08 | 2.24E−05 | 0.78 | 1.00 | −0.33 |
| rs4525161 | ADRA2A | 10 | 112992712 | 0.44 | 2.32E−05 | 0.78 | 1.00 | −0.34 |
| rs1343151 | IL23R | 1 | 67491717 | 0.34 | 2.59E−05 | 0.78 | 1.00 | −0.33 |
| rs1013895 | GLS | 2 | 191377084 | 0.17 | 2.73E−05 | 0.78 | 1.00 | 0.33 |
| rs10179858 | GLS | 2 | 191393971 | 0.17 | 2.73E−05 | 0.78 | 1.00 | 0.33 |
| rs10144421 | C14orf49 | 14 | 94981588 | 0.41 | 2.80E−05 | 0.78 | 1.00 | −0.33 |
| rs12070470 | IL23R | 1 | 67469859 | 0.07 | 3.00E−05 | 0.78 | 1.00 | 0.33 |
| rs17775850 | ADRA2A | 10 | 112984438 | 0.40 | 3.04E−05 | 0.78 | 1.00 | −0.33 |
| rs6502555 | GARNL4 | 17 | 2676402 | 0.33 | 3.63E−05 | 0.85 | 1.00 | 0.32 |
| rs860623 | SIPA1L3 | 19 | 43305194 | 0.22 | 3.86E−05 | 0.85 | 1.00 | 0.32 |
| rs1426936 | HPGD | 4 | 175621502 | 0.42 | 3.94E−05 | 0.85 | 1.00 | −0.32 |
| rs12189146 | PRLR | 5 | 35275643 | 0.21 | 4.36E−05 | 0.85 | 1.00 | −0.32 |
| rs4975274 | PHF17 | 4 | 129997526 | 0.27 | 4.43E−05 | 0.85 | 1.00 | 0.32 |
| rs9325634 | TMPRSS3 | 21 | 42691859 | 0.48 | 4.65E−05 | 0.85 | 1.00 | −0.32 |
| rs7984685 | USP12 | 13 | 26635031 | 0.46 | 4.69E−05 | 0.85 | 1.00 | −0.32 |
| rs644178 | CNTN5 | 11 | 97411048 | 0.36 | 4.79E−05 | 0.85 | 1.00 | −0.32 |
| rs10516503 | TACR3 | 4 | 104772736 | 0.07 | 5.16E−05 | 0.85 | 1.00 | 0.32 |
| rs1725489 | SIPA1L3 | 19 | 43269866 | 0.32 | 5.29E−05 | 0.85 | 1.00 | −0.32 |
| rs11749532 | FLJ90709 | 5 | 55038022 | 0.26 | 5.80E−05 | 0.85 | 1.00 | −0.32 |
| rs863020 | SIPA1L3 | 19 | 43304833 | 0.20 | 5.91E−05 | 0.85 | 1.00 | 0.31 |
| rs6768558 | TBL1XR1 | 3 | 178890973 | 0.19 | 5.95E−05 | 0.85 | 1.00 | 0.31 |
| rs511049 | PITX2 | 4 | 112047584 | 0.19 | 5.98E−05 | 0.85 | 1.00 | −0.31 |
| rs17503919 | RNGTT | 6 | 89622456 | 0.05 | 6.00E−05 | 0.85 | 1.00 | 0.31 |
| rs4824388 | AGTR2 | 23 | 115314659 | 0.21 | 6.10E−05 | 0.85 | 1.00 | −0.31 |
| rs2162171 | API5 | 11 | 43097900 | 0.14 | 6.41E−05 | 0.85 | 1.00 | 0.31 |
| rs1313925 | NFKB1 | 4 | 103596601 | 0.37 | 6.69E−05 | 0.85 | 1.00 | 0.31 |
| rs2064112 | NEDD9 | 6 | 11447755 | 0.25 | 6.70E−05 | 0.85 | 1.00 | −0.31 |
| rs7665426 | KIAA1712 | 4 | 175562261 | 0.26 | 6.81E−05 | 0.85 | 1.00 | −0.31 |
| rs346119 | AGA | 4 | 179920397 | 0.41 | 6.93E−05 | 0.85 | 1.00 | 0.31 |
| rs4563418 | C3orf38 | 3 | 88562476 | 0.25 | 6.95E−05 | 0.85 | 1.00 | 0.31 |
| rs16851554 | SPAG16 | 2 | 214732637 | 0.17 | 8.06E−05 | 0.95 | 1.00 | −0.31 |
| rs13361664 | PRLR | 5 | 35270600 | 0.17 | 8.32E−05 | 0.95 | 1.00 | −0.31 |
| rs12071280 | VAV3 | 1 | 108357521 | 0.13 | 9.38E−05 | 0.95 | 1.00 | −0.31 |
| rs12518171 | COX7C | 5 | 86165421 | 0.31 | 9.50E−05 | 0.95 | 1.00 | −0.31 |
| rs1111972 | OR1J2 | 9 | 124302127 | 0.06 | 9.81E−05 | 0.95 | 1.00 | −0.31 |

*false discovery rate,
^{†}Bonferroni corrected p-value,
^{§}Pearson's correlation coefficient.

Ingenuity pathway analysis also was performed to identify pathways and networks associated with NAPQI toxicity based on basal mRNA expression. All probe sets for which basal expression was associated with IC₅₀ with p<10⁻⁴ were used in this analysis. The top three “biological functions” of genes associated with NAPQI IC₅₀ were cell signaling, vitamin/mineral metabolism, and gene expression (p=8.4×10⁻⁴, 8.4×10⁻⁴, and 3.9×10⁻³, respectively). The top three canonical pathways were PXR/RXR activation (p=3.2×10⁻³), N-glycan biosynthesis (p=2.4×10⁻²), and glutamate receptor signaling (p=3.0×10⁻²).

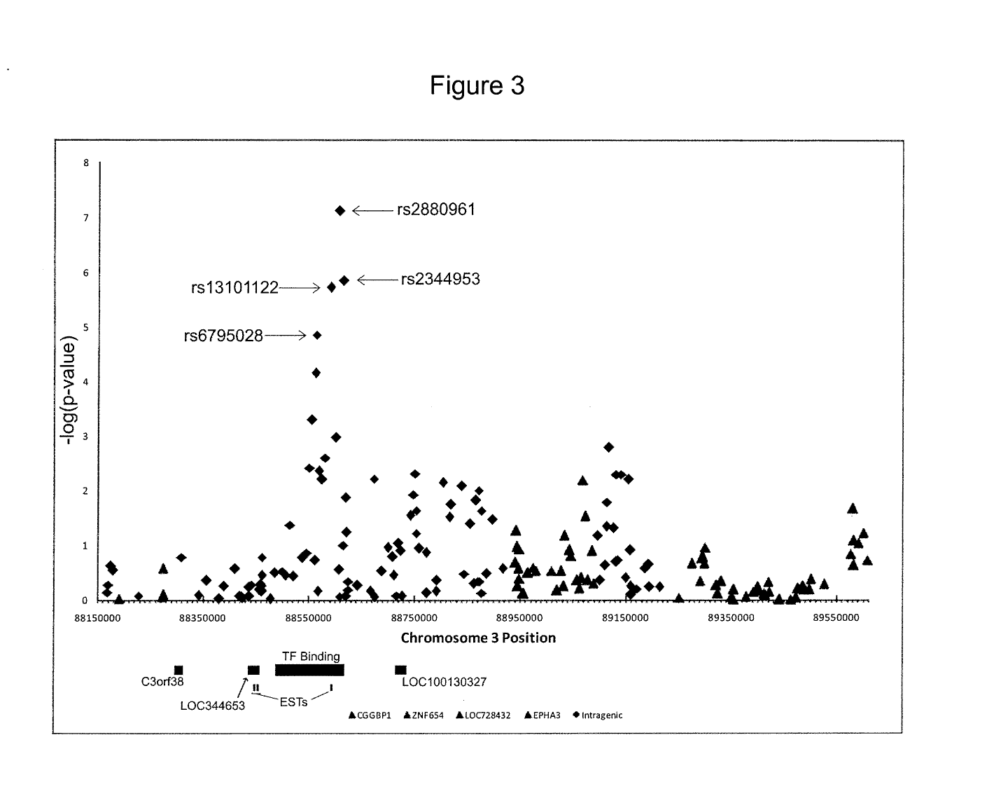

The genome-wide SNP association study identified a group of 4 SNPs (rs2880961, rs2344953, rs13101122, and rs6795028) that were highly associated with NAPQI toxicity (p=7.5×10⁻⁸, 1.42×10⁻⁶, 1.86×10⁻⁶, and 1.38×10⁻⁵, respectively) on Chromosome 3 (Table 4, FIG. 3). These 4 SNPs were in linkage disequilibrium (rs2880961/rs13101122, r²=0.92; rs2889061/rs2344953, r²=0.88; rs13101122/rs2344953, r²=0.80; rs6795028/rs2880961, r²=0.48; rs6795028/rs2344953, r²=0.44; and rs6795028/rs13101122, r²=0.41). Of these 4 SNPs, the most highly associated (rs2880961) was significantly associated with IC₅₀ even after the conservative Bonferroni correction for multiple comparisons (p=0.039). These SNPs are located in a region of Chromosome 3 that is distant from known genes, and is 275 kb downstream from C3orf38 and 624 kb upstream of EPHA3 (FIG. 3). Although this region is far from known genes, examination of this region with the Vista Genome Browser showed many evolutionarily conserved segments. One of those regions, which is predicted to be a portion of an unidentified gene, is located only about 7 kb upstream from the SNP with the strongest signal in the genome-wide SNP association study.

### Example 5

### Characterization of rs2880961

The conserved segments near the chromosome 3 SNPs associated with NAPQI IC₅₀ values may represent transcription factor binding sites. Therefore, a transcription factor binding site search, TFSEARCH (Heinemeyer et al. (1998) Nucl. Acids Res. 26:362-367), was performed to identify potential alterations in transcription factor binding by rs2880961. When the nucleotide present at that locus is the WT nucleotide (cytosine), only C/EBP is predicted to bind (score=86.2 out of 100). However, when the variant nucleotide (thymine) is present, binding sites are predicted to be introduced for HSF2 (score=90.4), NF-kappaB (89.6), and HSF1 (87.7), while the C/EBP binding site is predicted to remain (score=86.2).

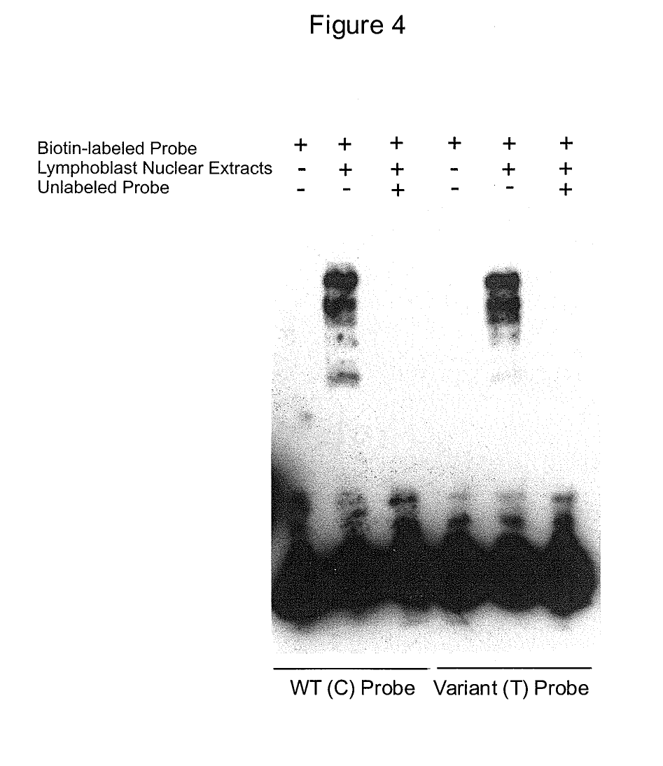

Because differential protein binding to the rs2880961 locus for the WT and variant nucleotide was predicted, an electrophoretic mobility shift assay (EMSA) was next performed in order to assess protein binding in the presence of both the WT and variant nucleotide. The binding pattern observed was similar between the WT and variant, but the intensity of one band was much stronger in the presence of the WT probe than the variant probe (FIG. 4). Although additional bands were not identified in the presence of the variant probe when compared to the WT probe (as expected based on the transcription factor binding prediction), the observed change in intensity suggested possible differential binding affinity between the WT and variant, which could result in differential transcription regulation.

If differential binding occurs between the WT and variant nucleotide at this locus, mRNA expression of other genes may be affected. Because there were no candidate genes located near this position that could be tested, a genome-wide association of genotype at rs2880961 with genome-wide mRNA expression probes was performed, and three probe sets were identified as being associated with rs2880961 genotype and having p<1×10⁻⁴. The most significantly associated probe set, 202132_at (p=3.7×10⁻⁵), corresponded to WWTR1 (also known as TAZ), which is a transcriptional regulator. Basal expression of this probe set was associated with IC₅₀, with p=0.00085. The other two probe sets, 207826_s_at (p=9.2×10⁻⁵) and 234741_at (p=9.6×10⁻⁵), corresponded to ID3 (inhibitor of DNA binding 3), and ATP2B2 (a Ca⁺⁺ transporting ATPase).

### Example 6

### Post-NAPQI mRNA Microarray Changes

The cellular response to NAPQI has not previously been assessed in terms of changes in mRNA expression. Therefore, to better understand cellular responses to NAPQI, mRNA expression post-NAPQI was compared to pre-exposure expression for three sensitive and three resistant lymphoblastoid cell lines, as well as HepG2 cells to model what expression changes might occur in the liver in response to NAPQI. Although both the sensitive and resistant lymphoblastoid cell lines were treated with the IC₅₀ specific to the cell line, the response was quite different in the sensitive and resistant cell lines. In the sensitive cell lines, the most dramatic changes in gene expression were approximately 3-fold changes. In the resistant cell lines, the most dramatic change in gene expression was a 700-fold increase, and many other transcripts were increased 100-fold. In addition to the sensitive cell lines demonstrating smaller changes in expression after exposure to NAPQI, the canonical pathways involved differed between the sensitive and resistant lymphoblasts. Upon Ingenuity Pathway analysis, the resistant cells were found to have changes in p53 signaling (p=7.9×10⁻⁵), IL-10 signaling (p=3.3×10⁻⁵), and G2/M DNA damage checkpoint regulation (p=3.4×10⁻⁵). The sensitive cells had changes in G1/S checkpoint regulation (p=5.1×10⁻⁴), G2/M DNA damage checkpoint regulation (p=5.4×10⁻³), and the protein ubiquitination pathway (p=1.6×10⁻²).

Ingenuity Pathway analysis also was performed to determine patterns of altered gene transcription after exposure of HepG2 cells to NAPQI. HepG2 cells were incubated with a high dose of NAPQI to model an acute overdose. In this system, the most dramatic changes in transcription were approximately 20-fold. The top canonical pathways altered by NAPQI exposure were biosynthesis of steroids (p=7.0×10⁻⁸), role of BRCA1 in DNA damage response (p=1.2×10⁻⁷), G2/M DNA damage checkpoint regulation (p=1.2×10⁻⁵), hepatic fibrosis/hepatic stellate cell activation (p=4.9×10⁻⁴), and p53 signaling (p=5.9×10⁻⁴). “Cellular growth and proliferation” was the category with the highest representation (63 molecules) of the molecular function categories analyzed with Ingenuity for changes in expression after NAPQI.

### Example 7

### Clinical Validation Study Analysis

Both race and gender were found to have an effect on ALT levels (p=0.046 and p=0.0004, respectively), but did not affect the change in ALT in response to acetaminophen (p=0.32 and p=0.23, respectively). The analysis for association between rs2880961 genotype and ALT was therefore adjusted for race and gender. Males in the study generally had higher ALT values than females, both at baseline and after APAP treatment. After adjustment for gender and race, baseline ALT was associated with genotype rs2880961, p=0.047. rs2880961 was not associated with change in ALT after acetaminophen administration, however.

Although the mechanism by which the identified SNP signal may be associated with NAPQI toxicity remains unclear, an attempt to validate the signal in a clinical population was made. For these studies, the therapeutic parent compound acetaminophen was used rather than the highly reactive and toxic NAPQI. Despite the small number of subjects, the study revealed an interesting association. While rs2880961 was not associated with change in ALT after acetaminophen administration as expected, it was associated with baseline ALT values. This observation suggested that the identified SNP signal could be related to general susceptibility of the liver to toxicity, rather than to one particular toxin, acetaminophen.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for predicting the likelihood of acetaminophen toxicity in a subject, said method comprising:
(a) determining whether a biological sample from said subject comprises a wild type or variant rs2880961 allele, and
(b) classifying said subject as having a greater likelihood of acetaminophen toxicity if said variant allele is present in said biological sample, and classifying said subject as having a lesser likelihood of acetaminophen toxicity if said wild type allele is present in said biological sample.

2. The method of claim 1, wherein said subject is a human.

3. A method for determining a tolerable dose of acetaminophen for administration to a subject, said method comprising:
(a) determining whether a biological sample from said subject comprises a wild type or variant rs2880961 allele, and
(b) determining that said tolerable dose is lower if said variant allele is present in said biological sample, and determining that said tolerable dose is higher if said wild type allele is present in said biological sample.

4. The method of claim 1, wherein said subject is a human.

5. A method of assessing likelihood of acetaminophen toxicity in a subject, said method comprising:
(a) receiving a biological sample obtained from said subject,
(b) assaying said sample to determine whether said sample comprises a wild type or variant rs2880961 allele,
(c) communicating to a medical or research professional information about whether said wild type or variant allele is present in said sample, and
(d) before or after step (a), communicating to a medical or research professional information indicating that the presence of said variant allele correlates with acetaminophen toxicity.

6. The method of claim 5, wherein said subject is human.

7. A method for determining a tolerable dose of acetaminophen for administration to a subject, said method comprising:
(a) receiving a biological sample obtained from said subject,
(b) assaying said sample to determine whether said sample comprises a wild type or variant rs2880961 allele,
(c) communicating to a medical or research professional information about whether said wild type or variant allele is present in said sample, and
(d) before or after step (a), communicating to a medical or research professional information indicating that the presence of said variant allele correlates with a lower suggested dose.

8. The method of claim 7, wherein said subject is human.
